(19) United States
(12) Patent Application Publication
(10) Pub. No.: US 2014/0024891 A1
(43) Pub. Date: Jan. 23, 2014

(54) ENDOSCOPE SYSTEM AND IMAGE PLAYBACK METHOD IN ENDOSCOPE SYSTEM

(71) Applicant: OLYMPUS CORPORATION, Tokyo (JP)

(72) Inventor: Ryoji MOTOKI, Yokohama-shi (JP)

(73) Assignee: OLYMPUS CORPORATION, Tokyo (JP)

(21) Appl. No.: 13/709,367

(22) Filed: Dec. 10, 2012

(30) Foreign Application Priority Data
Jul. 18, 2012 (JP) 2012-159867

(51) Int. Cl. A61B 1/00; A61B 1/04

(52) U.S. Cl. CPC A61B 1/00045; A61B 1/04; A61B 1/0002
USPC 600/103

(57) ABSTRACT

An endoscope apparatus detects input of a position on a moving endoscope image to be recorded, stores a piece of coordinate information of the detected position in association with each of frames of the moving endoscope image, reads out pieces of frame data of the moving image from a memory card which records the pieces of frame data of the moving image, judges whether each of the frames is associated with the piece of coordinate information, superimposes an indicator M2 at the position on the basis of the piece of coordinate information if the frame is associated with the piece of coordinate information, and plays back the moving endoscope image.

### CROSS REFERENCE TO RELATED APPLICATION

This application claims benefit of Japanese Application No. 2012-159867 filed in Japan on Jul. 18, 2012, the contents of which are incorporated by this reference.

### BACKGROUND OF THE INVENTION

1. Field of the Invention

The present invention relates to an endoscope system and an image playback method in the endoscope system and, more particularly, to an endoscope system capable of efficiently confirming an inspection result and an image playback method in the endoscope system.

2. Description of the Related Art

Endoscope apparatuses have been widely used in a medical field and an industrial field. An endoscope apparatus includes an insertion portion which is to be inserted into an object to be observed, and the insertion portion has an image pickup device provided at, e.g., a distal end portion. In the industrial field, the endoscope apparatus is used to observe and inspect flaws and corrosion in an interior of piping, a boiler, a turbine, an engine, or the like by inserting the insertion portion into the piping or the like.

An endoscope apparatus not only can display an endoscope image of a site to be observed in an inspection target on a monitor during inspection but also can record an endoscope image as a still image or a moving image. In the case, the endoscope image is recorded in a storage device inside the endoscope apparatus or a recording medium removable from the endoscope apparatus.

A recorded endoscope image can be played back in an endoscope apparatus or in an external device such as a personal computer (hereinafter abbreviated as PC) in order to confirm status of a site to be inspected after inspection.

For example, if a flaw or the like is found at the time of endoscopy, a user of an endoscope apparatus serving as an inspector may record an endoscope image showing the flaw or the like as a moving image. In the case, the user can take and record an endoscope image as a moving image and then confirm an inspection result while playing back the recorded moving image.

As disclosed in Japanese Patent Application Laid-Open Publication No. 2005-341241, an image recording and playback apparatus is proposed which generates and records information on a point of interest in each frame at the time of recording an image for generation of a trimmed image. According to the image recording and playback apparatus, at the time of playback, a part of the image including points of interest is cut out and is outputted as trimmed image data.

### SUMMARY OF THE INVENTION

An endoscope system according to one aspect of the present invention includes a position input detection portion which detects input of a position on a moving endoscope image to be recorded, a coordinate information storage portion which stores a piece of coordinate information of the position detected by the position input detection portion in association with each of frames of the moving endoscope image, a recording portion which records pieces of frame data of the moving image, and a moving image playback portion which reads out the pieces of frame data of the moving image from the recording portion, judges whether each of the frames is associated with the piece of coordinate information, superimposes a predetermined first mark at the position on the basis of the piece of coordinate information if the frame is associated with the piece of coordinate information, and plays back the moving endoscope image.

### BRIEF DESCRIPTION OF THE DRAWINGS

FIG. 1 is a block diagram showing a configuration of an endoscope apparatus according to an embodiment of the present invention;

FIG. 2 is a flow chart showing an example of a flow of a moving image recording process according to the embodiment of the present invention;

FIG. 3 is a view for explaining an example of a screen of an LCD 4 when a user touches the screen with a finger at the time of recording a moving image, according to the embodiment of the present invention;

FIG. 4 is a flow chart showing an example of a flow of a moving image playback process according to the embodiment of the present invention;

FIG. 5 is a view for explaining an example of a screen at the time of playing back a recorded moving image according to the embodiment of the present invention;

FIG. 6 is a chart for explaining a skip playback function according to the embodiment of the present invention;

FIG. 7 is a view showing an example of a playback position display bar which is displayed together with a moving image at the time of playback, according to the embodiment of the present invention;

FIG. 8 is a flow chart showing an example of a flow of a moving image playback process according to a first modification of the embodiment of the present invention;

FIG. 9 is a flow chart showing an example of a flow of a moving image playback process according to a second modification of the embodiment of the present invention;

FIG. 10 is a flow chart showing an example of a flow of a moving image playback process according to a third modification of the embodiment of the present invention;

FIG. 11 is a flow chart showing an example of a flow of a moving image playback process according to a fourth modification of the embodiment of the present invention;

FIG. 12 is a diagram for explaining association between frame data and coordinate information, according to a sixth modification of the embodiment of the present invention; and

FIG. 13 is a flow chart showing an example of a flow of a moving image recording process according to a seventh modification of the embodiment of the present invention.

### DETAILED DESCRIPTION OF THE PREFERRED EMBODIMENT

An embodiment of the present invention will be described below with reference to the drawings.

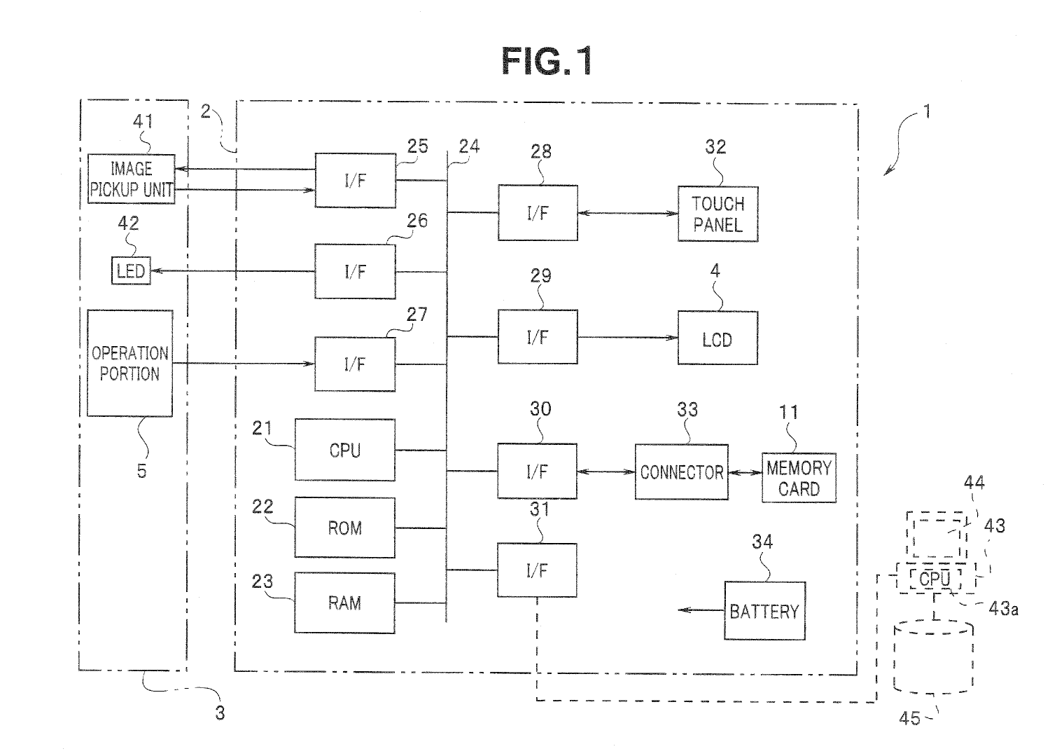

FIG. 1 is a block diagram showing a configuration of an endoscope apparatus according to the present embodiment. An endoscope apparatus 1 serving as an endoscope system is composed of a main body portion 2 serving as a main unit and a scope unit 3 which is connected to the main body portion 2. The main body portion 2 includes a liquid crystal panel (hereinafter abbreviated as LCD) 4 as a display device on which an endoscope image, an operation menu, and the like are displayed. The scope unit 3 has an insertion portion. The scope unit 3 and the main body portion 2 are connected by a connection cable.

The scope unit 3 further has an operation portion 5. A user can perform recording of a still image or a moving image of an object and other processing by operating various operation buttons of the operation portion 5. The operation portion 5 is provided with operation buttons for various functions, such as a freeze button and a record instruction button (hereinafter referred to as a REC button).

A user as an inspector brings a distal end portion of the insertion portion close to a site to be inspected of an inspection target. The user obtains an endoscope image by photographing the site to be inspected. The endoscope image is displayed on the LCD 4 and is optionally recorded on a memory card 11 serving as a recording medium. The memory card 11 is removable from the main body portion 2.

Note that although folders and pieces of image data are recorded on the memory card 11 as a recording medium removable from the main body portion 2 in the present embodiment, the folders and pieces of image data may also be recorded in an internal memory of the main body portion 2.

The main body portion 2 includes a central processing unit (hereinafter referred to as a CPU) 21, a ROM 22, and a RAM 23, all of which are connected to each other via a bus 24. Various interfaces (hereinafter referred to as I/Fs) 25 to 31 are further connected to the bus 24.

The I/F 25 is a driver and receiver circuit for transmitting a driving signal from the scope unit 3 to an image pickup unit 41 and receiving an image pickup signal from the image pickup unit 41.

The image pickup unit 41 is an image pickup portion which is composed of an image pickup device such as a CCD sensor or a CMOS sensor and an image pickup optical system such as a lens arranged on the side of an image pickup surface of the image pickup device.

The I/F 26 is a driver circuit for transmitting a driving signal to an LED 42 as an illumination portion.

The I/F 27 is a circuit for receiving various operation signals from the operation portion 5.

The LCD 4 is provided with a touch panel 32. The I/F 28 is a circuit for receiving a driving signal to the touch panel 32 and an operation signal from the touch panel 32. The I/F 29 is a circuit for supplying an image signal to the LCD 4.

The I/F 30 is a circuit for writing an image signal to the memory card 11 and reading out an image signal from the memory card 11. The I/F 30 is connected to the memory card 11 via a connector 33 which is provided in the main body portion 2. The memory card 11 is removably installed in the connector 33.

The I/F 31 is a circuit for connecting a PC 43 serving as an external device to the main body portion 2. The PC 43 is connected to the main body portion 2 via a connector (not shown). The main body portion 2 can exchange data with the PC 43 via the I/F 31 connected to the connector.

As indicated by dotted lines, the PC 43 has a CPU 43a and a monitor 44. A storage device 45 is connected to the PC 43. The PC 43 further has a connector for a memory card (not shown). A user can install the memory card 11 in the PC 43 and display an endoscope image stored in the memory card 11 on the monitor 44 or transfer the endoscope image to the storage device 45 for storage in the storage device 45.

The main body portion 2 incorporates a battery 34. The battery 34 supplies power to the various circuits in the main body portion 2.

Each I/F operates under control of the CPU 21. When the endoscope apparatus 1 is activated, the CPU 21 outputs various driving signals to the image pickup unit 41 via the I/F 25. The image pickup unit 41 outputs an image pickup signal to the CPU 21. The CPU 21 outputs a drive instruction signal for the LED 42 to the I/F 26, and the LED 42 is driven by an output from the I/F 26. As a result, an endoscope image of an object irradiated with illuminating light from the LED 42 is displayed on the LCD 4.

The operation portion 5 is connected to the CPU 21 via the I/F 27. The operation portion 5 supplies various operation signals indicating details of a user's operation on the operation portion 5 to the CPU 21. When a user operates a predetermined button, the CPU 21 can record a still image or a moving image on the memory card 11 on the basis of an image pickup signal from the image pickup unit 41. A user can also display an image recorded on the memory card 11 on the LCD 4 by operating the operation portion 5.

Various programs corresponding to various modes are stored in the ROM 22 of the endoscope apparatus 1. The CPU 21 can read out and execute a program corresponding to an instruction from a user as an inspector in accordance with the instruction. The endoscope apparatus 1 has various other modes in addition to endoscopy mode that is a mode when normal endoscopy is performed. One of the plurality of modes is spot-of-interest recording mode in which a predetermined mark is displayed and stored at the time of recording a moving image that is an endoscope image. As will be described below, if spot-of-interest recording mode is set at the time of moving image recording, a user can include position information of a spot of interest in moving image data and record the moving image data on the memory card 11.

### (Moving Image Recording Process)

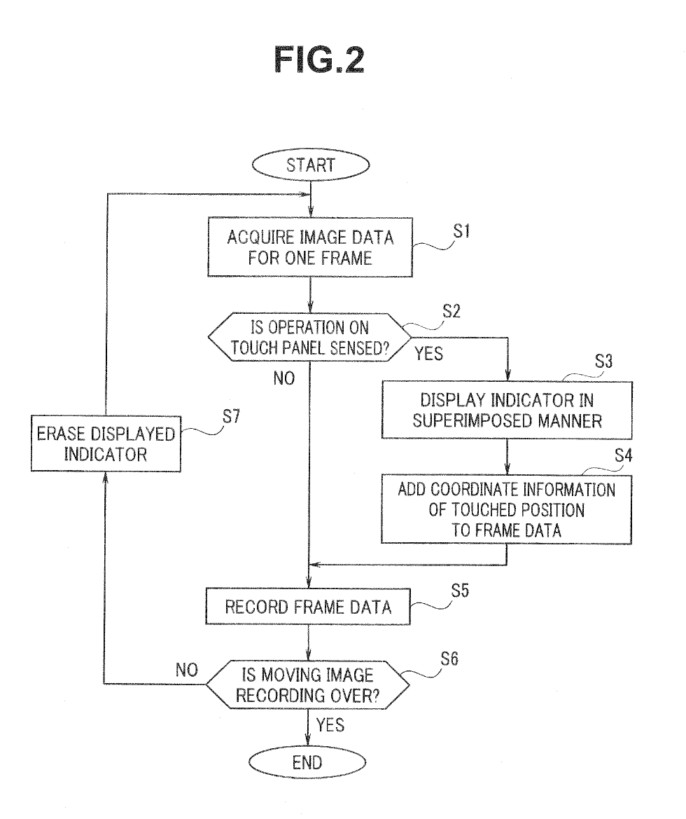

FIG. 2 is a flow chart showing an example of a flow of a moving image recording process.

If instructions to record a moving image are given when the endoscope apparatus 1 is set to spot-of-interest recording mode, and a user is performing endoscopy with an endoscope image displayed on the LCD 4, the process in FIG. 2 is executed. The process in FIG. 2 is executed by the CPU 21. An endoscope image based on image pickup signals outputted from the image pickup unit 41 when displayed substantially in real time is referred to as a live image here.

When the moving image recording process is started, the CPU 21 acquires a piece of image data for one frame (i.e., a piece of frame data) from image pickup signals outputted from the image pickup unit 41 (S1).

When the CPU 21 acquires the piece of image data for one frame, the CPU 21 judges (S2) whether an operation on the touch panel 32, i.e., a touch is sensed. The judgment in S2 can be made by receiving an operation signal from the touch panel 32. The process in S2 constitutes a position input detection portion which detects input of a position on a moving endoscope image to be recorded.

If an operation on the touch panel 32 is sensed (YES in S2), the CPU 21 displays an indicator M1 as a predetermined mark in a superimposed manner on the touch panel 32, i.e., at a touched position on a screen of the LCD 4 (S3). The process in S3 constitutes an endoscope image display portion which displays the predetermined mark M1 at the position detected in S2 so as to be superimposed on the moving endoscope image to be recorded on the basis of a piece of coordinate information of the position.

The CPU 21 adds the piece of coordinate information of the touched position detected in S2 to the piece of frame data (S4). The piece of coordinate information of the touched position is included in the operation signal from the touch panel 32.

As a format of moving image data, various formats are available. The addition of a piece of coordinate information to a piece of frame data can be performed by, for example, writing the piece of coordinate information to a piece of header information corresponding to the piece of frame data. Note that a piece of coordinate information may be stored in association with a frame number in a file different from a file for frame data.

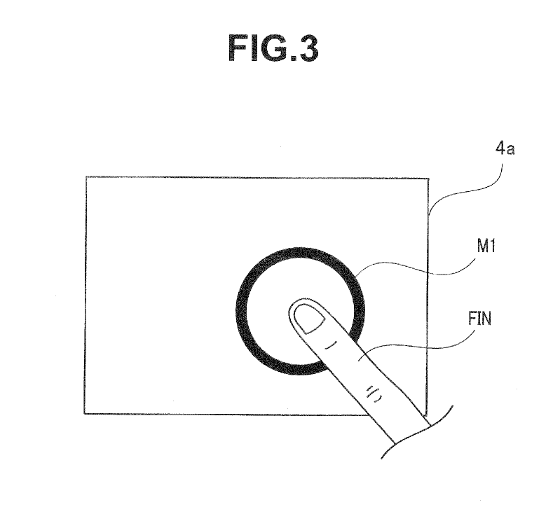

FIG. 3 is a view for explaining an example of a screen of the LCD 4 when a user touches the screen with the user's finger at the time of moving image recording. When the user touches the screen of the LCD 4 with a finger FIN, the touch panel 32 outputs a piece of coordinate information of a touched position to the CPU 21 via the I/F 28.

The CPU 21 displays the circular indicator M1 as shown in FIG. 3 at the position touched by the finger FIN (S3). The indicator M1 is a circular figure having the touched coordinate position on the screen at a center and having a predetermined radius.

Note that although the indicator M1 is a circular figure having a predetermined diameter in FIG. 3, the indicator M1 may be a figure of any other shape, such as a rectangle, a point, an arrow, or an animation.

If an operation on the touch panel 32 is not sensed (NO in S2) or after the piece of coordinate information is added to the piece of frame data (S4), the CPU 21 executes a process of recording the piece of frame data (S5). In the process in S5, a piece of image data for each frame is written to the memory card 11 as a recording medium. The processes in S4 and S5 constitute a coordinate information storage portion which stores a piece of coordinate information of a position detected in S2 in association with each frame of a moving endoscope image.

It is judged (S6) whether instructions to end moving image recording are given. Instructions to end moving image recording are given by a user's performing a predetermined operation on the operation portion 5. The CPU 21 makes the judgment in S6 on the basis of presence or absence of the predetermined operation.

If instructions to end moving image recording are not given (NO in S6), the CPU 21 erases the displayed indicator M1 (S7), and the process shifts to S1 to execute the above-described processes for a next frame.

If instructions to end moving image recording are given (YES in S6), the moving image recording process ends.

Accordingly, while a touch on the touch panel 32 is being detected, a piece of coordinate information is added for each frame of a moving image and is recorded on the memory card 11, and the predetermined indicator M1 is displayed on a touched screen of the LCD 4. The memory card 11 is a recording portion which records pieces of frame data of a moving image. In S5, a piece of coordinate information is stored in the memory card 11 serving as the recording portion that records a moving endoscope image. At the time, the piece of coordinate information is stored in association with each frame by being written into a piece of frame data of the frame.

Note that although pieces of coordinate information are stored so as to correspond to respective frames in the above example, if there is no change or substantially no change between sensed pieces of coordinate information, a piece of coordinate information may be stored in association only with a first frame, and a piece of coordinate information may not be stored in association with a frame subsequent to the first frame.

### (Playback Process)

If spot-of-interest display mode is set at the time of playing back a moving image that is an endoscope image, a predetermined mark is displayed in a superimposed manner on the basis of a piece of coordinate information of a position recorded at the time of recording.

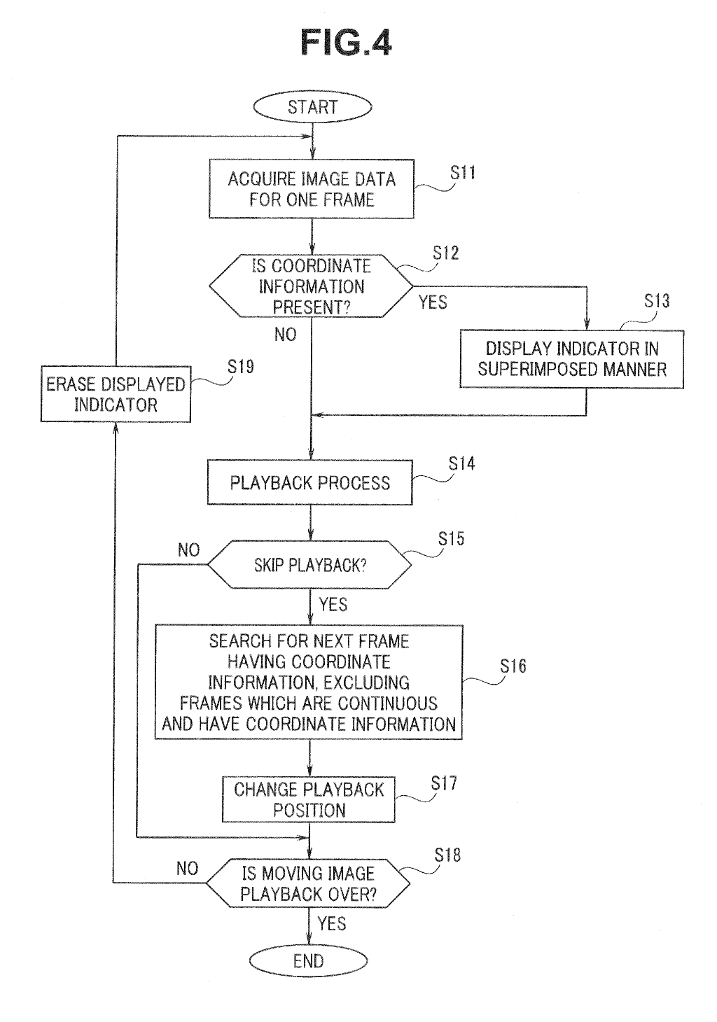

FIG. 4 is a flow chart showing an example of a flow of a moving image playback process.

A moving image recorded on the memory card 11 can be played back by the endoscope apparatus 1. When the endoscope apparatus 1 is set to spot-of-interest display mode, and a user gives instructions for the moving image playback process by inputting predetermined instructions, the process in FIG. 4 is executed. The process in FIG. 4 is executed by the CPU 21.

When the playback process is started, the CPU 21 acquires a piece of image data for one frame (i.e., a piece of frame data) of a moving image of a file designated to be played back from the memory card 11 (S11).

The CPU 21 judges (S12) whether there is a piece of coordinate information associated with the frame. In the above example, a piece of coordinate information is added to each piece of frame data at the time of moving image recording. The CPU 21 can make the judgment in S12 by detecting whether a piece of coordinate information is included in the acquired piece of frame data.

If there is a piece of coordinate information associated with the acquired frame (YES in S12), the CPU 21 displays an indicator M2 in a superimposed manner at a position designated by the piece of coordinate information (S13).

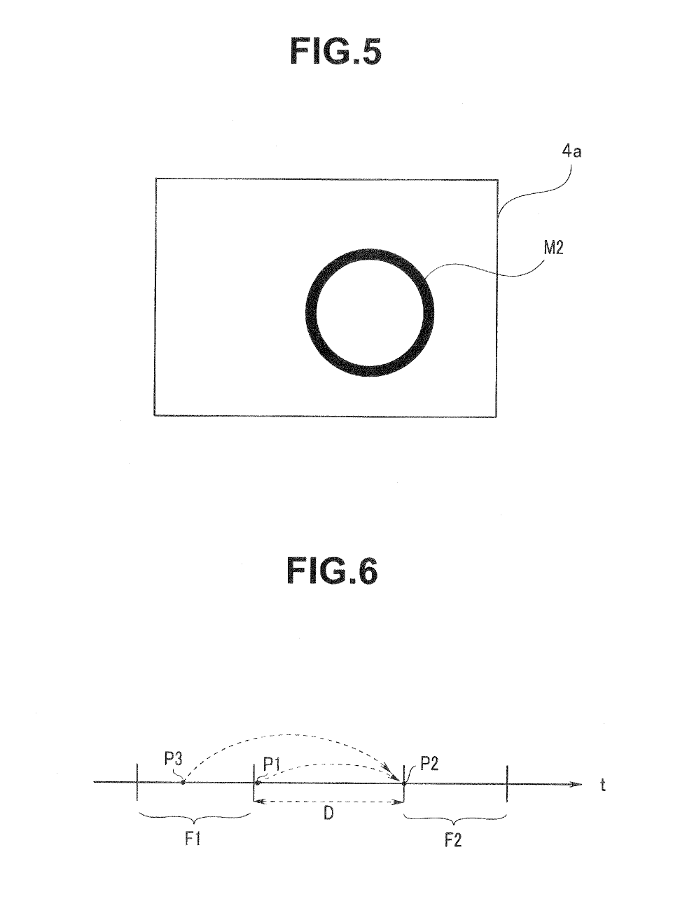

FIG. 5 is a view for explaining an example of a screen at the time of playing back a recorded moving image. If there is a piece of coordinate information associated with the acquired frame (YES in S12), the CPU 21 displays the circular indicator M2 as a predetermined mark as shown in FIG. 5 in a superimposed manner at the position designated by the piece of coordinate information. In the present embodiment, the indicator M2 is identical to the indicator M1 described above. That is, the indicator M2 is a circular figure having the coordinate position at a center and having the predetermined radius.

As described above, the processes in S12 to S15 constitute a moving image playback portion which reads out a piece of frame data of a moving image from the memory card 11 serving as the recording portion, judges whether a piece of coordinate information is associated with each frame, superimposes the predetermined mark M2 at a position based on a piece of coordinate information for the frame if the frame is associated with the piece of coordinate information, and plays back the moving endoscope image.

Note that a shape and a color of the indicator M2 at the time of playback may be different from a shape and a color of the indicator M1 at the time of recording. Although the indicator M2 is also a circular figure having the predetermined diameter in FIG. 5, the indicator M2 may be a figure of any other shape, such as a rectangle, a point, or an animation.

If there is no piece of coordinate information for the acquired frame (NO in S12) or after the process of displaying the indicator M2 (S13), a playback process is executed (S14), and presence or absence of instructions for skip playback is judged (S15). Accordingly, the moving endoscope image is played back while the indicator M2 serving as the predetermined mark is not superimposed for the frame if the frame is not associated with a piece of coordinate information.

Various command buttons are displayed in a playback screen. As one of the command buttons, a button for giving instructions for skip playback is provided. When a user selects the skip playback button (i.e., designates the button with a cursor) on a screen 4a of the LCD 4, a playback position skips. With the instruction for skip playback in S15, a process of changing the playback position from a current playback position to a position of a frame which has a piece of coordinate information next and resuming playback is executed.

FIG. 6 is a chart for explaining a skip playback function. FIG. 6 shows that there are two groups F1 and F2 of pieces of frame data with attached pieces of coordinate information along passage of a playback time t. The two groups F1 and F2 of pieces of frame data are separated by a time interval D.

If a user gives instructions for skip playback when a playback position is at a position P1, and a moving image being played back is displayed on the LCD 4, the playback position is changed from the position P1 to a position P2 at a beginning of the group F2 of pieces of frame data with the attached piece of coordinate information. Accordingly, the playback position jumps from P1 to P2, and the moving image is played back from the position P2. Since a part from the position P1 to the position P2 is a frame(s) with no piece of coordinate information, the user can skip the frame(s) with no piece of coordinate information after the position P1 without playing back the frame(s) and play back the moving image from the position P2 of a frame with a piece of coordinate information.

If the user gives instructions for skip playback when the playback position is at a position P3, and the moving image being played back is displayed on the LCD 4, the playback position is changed from the position P3 to the position P2 at the beginning of the group F2 of pieces of frame data which are the next to have a piece of coordinate information among frames after the position P3, excluding the group F1 of pieces of frame data for frames which have respective pieces of coordinate information in a row.

Referring back to FIG. 4, if instructions for skip playback are given in S14 (YES in S15), the CPU 21 searches (S16) for a frame which has a piece of coordinate information next among frames, excluding frames which are continuous with the current frame and have coordinate information like the current frame. More specifically, when the CPU 21 senses a press of the skip playback button, in S16, the CPU 21 sequentially reads out pieces of frame data after the current playback position in the moving image file being played back in the memory card 11 and checks presence or absence of a piece of coordinate information. In the case of the example in FIG. 6, a process of searching for the position P2 when the playback position is at the position P1 or the position P3 is performed in S16.

The CPU 21 changes the playback position to a position of a searched and retrieved frame with a piece of coordinate information (S17). That is, when a frame with a piece of coordinate information is detected as a result of checking presence or absence of a piece of frame data with a piece of coordinate information in S16, the playback position is changed to a position of the detected frame.

As described above, in S15 to S17, if a skip playback command is inputted at the time of playing back a recorded moving image, a frame associated with a piece of coordinate information is searched for among frames later than a frame at the current playback position, and the playback position is changed from the current playback position to a position of the frame associated with a piece of coordinate information that is detected by the search.

Note that if instructions for skip playback are not given in S15 (NO in S15), the process shifts to S18.

It is judged (S18) whether instructions to end moving image playback are given. Instructions to end moving image playback are given by a user performing a predetermined operation on the operation portion 5. The CPU 21 makes the judgment in S18 on the basis of presence or absence of the predetermined operation.

If instructions to end moving image playback are not given (NO in S18), and when the indicator is displayed, the displayed indicator is erased (S19).

As described above, in the moving image recording process, while a touch on the touch panel 32 is detected, a piece of coordinate information of a touched position (i.e., a position designated as a spot of interest by a user) is added or attached to each piece of frame data, and the indicator M1 is displayed at the coordinate position. When a touch on the touch panel 32 is not detected, each piece of frame data is recorded without change (i.e., a piece of coordinate information is not added to each piece of frame data).

Additionally, in the moving image playback process, if a read-out and acquired frame has an added piece of coordinate information, a frame image is displayed with the indicator M2 serving as a landmark of a spot of interest superimposed at a corresponding coordinate position. If the read-out and acquired frame does not have an added piece of coordinate information, a frame image is displayed without displaying the indicator M2.

Moreover, skip playback allows omitting playing back a piece of frame data with no piece of coordinate information, i.e., skipping the piece of frame data and playing back a piece of frame data which has a piece of coordinate information next.

In particular, since only pieces of frame data with pieces of coordinate information can be displayed in sequence by repeatedly pressing the skip playback button, a time period for checking content to be inspected of a moving image can be largely shortened.

As described above, a user can designate a spot of interest at the time of recording a moving image. When the moving image is played back, since a mark indicating a designated spot of interest is displayed together with the moving image, the user can easily find a spot of interest after inspection.

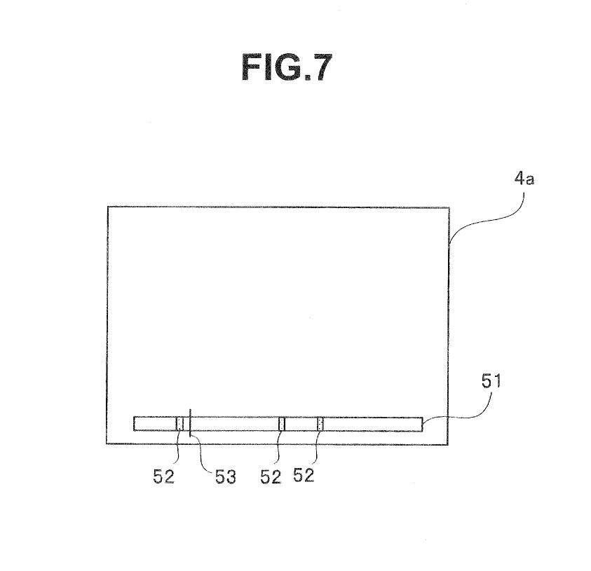

FIG. 7 is a view showing an example of a playback position display bar which is displayed together with a displayed moving image at the time of playback. A playback position display bar 51 is displayed on a lower side of the screen 4a of the LCD 4.

Upon receipt of instructions to play back a moving image, the CPU 21 first acquires all of pieces of time information for pieces of frame data with attached pieces of coordinate information among all pieces of frame data of the moving image, for which instructions for playback are given. The CPU 21 generates the playback position display bar 51 on the basis of all of pieces of time information for all the acquired pieces of frame data and changes a color of a position corresponding to a frame with a piece of coordinate information in the playback position display bar 51 to a color different from a background color. As a result, a point-of-interest indication portion 52 which indicates a part including a moving image in which a spot of interest is designated is displayed in an identifiable manner on the playback position display bar 51.

After the above-described playback position display bar 51 is displayed, the moving image playback process shown in FIG. 4 is executed.

In the playback position display bar 51 shown in FIG. 7, a playback position indication mark 53 allows a user to recognize a position being played back. If the user moves the playback position indication mark 53 using a cursor, a moving image is played back from a movement destination position. During playback of a moving image, the CPU 21 updates display of the playback position display bar 51 such that a current playback position of the moving image is always indicated by the playback position indication mark 53.

Accordingly, a user can know, at a glance, in which time segment of a moving image a spot of interest is present by the point-of-interest indication portion 52 displayed on the playback position display bar 51 during playback of the moving image without actually playing back a piece of frame data with an attached piece of coordinate information and can shorten a time period for inspection confirmation by playing back only a part in a time segment in which a spot of interest is present.

Note that although a position of contact with the touch panel 32 is designated as a position on a screen in the above-described embodiment, a position on a screen may be designated not by a touch panel but by a pointing device such as a mouse or a joystick.

Also, note that although playback of a moving image is performed in the endoscope apparatus 1 in the above-described embodiment, the above-described moving image playback process can be executed by installing the memory card 11 into the PC 43 and executing a program capable of executing the process shown in FIG. 4 by the CPU 43a in the PC 43. In the case, the endoscope apparatus 1 and the PC 43 constitute an endoscope system. The program for the process in FIG. 4 is stored in the storage device 45 and is read out and executed by the CPU 43a.

Although one spot of interest can be designated in the above-described embodiment, a plurality of spots of interest may be designated. For example, a plurality of positions can be designated by simultaneously touching a screen of the touch panel 32 with a plurality of fingers. In the case, a plurality of pieces of coordinate information of detected positions can be stored in association with each frame of a moving endoscope image in the processes in S4 and S5.

Note that although, in the moving image playback described above, the indicator M2 is displayed when a current frame is associated with a piece of coordinate information, the indicator M2 may be displayed only for a preset time period from display of a first frame if there is no change among pieces of coordinate information.

As described above, according to the endoscope system of the embodiment, a piece of coordinate information of a position of a spot of interest can be recorded in recording of a moving image that is an endoscope image.

Additionally, at the time of playback of a moving endoscope image, a predetermined mark is displayed on the basis of a piece of coordinate information of a position of a spot of interest on the endoscope image which is inputted at the time of moving image recording such that the spot of interest can be easily recognized. The configuration allows prevention of a user from missing a spot of interest.

Modifications of the above-described embodiment will be described.

### First Modification

In a first modification, when a piece of frame data associated with a piece of coordinate information starts to be played back at the time of playback, the playback is paused. The pause prevents an image with a spot of interest from being missed.

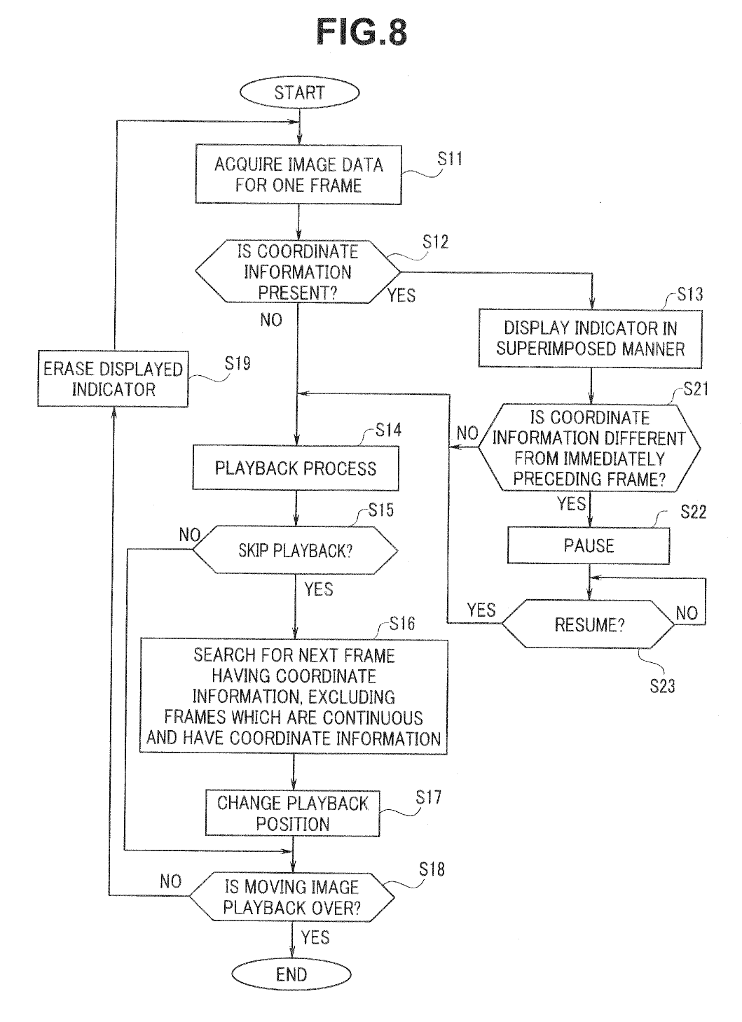

FIG. 8 is a flow chart showing a flow of a moving image playback process according to the first modification. In FIG. 8, same processes as the processes in FIG. 4 are denoted by same step numbers, and a description of the steps will be omitted. As shown in FIG. 4, after the indicator M2 is displayed so as to be superimposed on the piece of frame data associated with the piece of coordinate information, it is judged (S21) whether the piece of coordinate information is different from a piece of coordinate information of an immediately preceding piece of frame data. A case where the piece of coordinate information in S21 is different from the piece of coordinate information of the immediately preceding piece of frame data includes a case where the current frame is associated with a piece of coordinate information, and the immediately preceding piece of frame data is not associated with a piece of coordinate information.

If the piece of coordinate information is different from the piece of coordinate information of the immediately preceding piece of frame data (the case includes a case where an immediately preceding frame is not associated with a piece of coordinate information of a spot of interest, and the current frame is associated with a piece of coordinate information) (YES in S21), the CPU 21 pauses playback of the moving image (S22). In the paused state, a still image with the superimposed indicator M2 is displayed on the LCD 4.

At the time of the pause, playback of the moving image is paused, and a still image for the frame different in coordinate information from the immediately preceding frame is displayed. When a user gives instructions to resume playback to the CPU 21, the CPU 21 executes playback from the piece of frame data different in coordinate information from the immediately preceding piece of frame data.

In S23, the CPU 21 judges (S23) whether instructions to resume playback are given. If instructions to resume playback are given (YES in S23), the process shifts to S14. If instructions to resume playback are not given (NO in S23), no processing is performed.

As described above, in the processes in S13 to S14, if there is a shift from playback of a frame not associated with a piece of coordinate information to playback of a frame associated with a piece of coordinate information during playback of a recorded moving image, playback of the image is paused. When instructions to resume playback are given, playback is started from the paused frame associated with a piece of coordinate information.

Accordingly, even if a user takes the user's eyes from the LCD 4, playback of a moving image is paused immediately after the start of playback of an image including a spot of interest, and the user can view the moving image from the image with the spot of interest by giving instruction to resume playback. The user does not miss a spot of interest. Additionally, even if a current frame and an immediately preceding frame both have a piece of coordinate information and are different in a position of a spot of interest, playback is paused, and a user can view a moving image from an image with a spot of interest, a position of which is shifted, by giving instruction to resume playback. The user does not miss a spot of interest.

Note that although it is judged in S21 whether the piece of coordinate information of the acquired frame is different from the piece of coordinate information of the immediately preceding frame, if the pieces of coordinate information are little different (e.g., a difference between the pieces of coordinate information is not more than a predetermined value), i.e., two sets of coordinates are substantially same, the two pieces of coordinate information may be regarded as not different. This is to prevent a pause due to a slight difference.

### Second Modification

In a second modification, when a piece of frame data associated with a piece of coordinate information starts to be played back at the time of playback, a playback position is returned to a position earlier by a predetermined time period, and playback is performed from the position. The configuration prevents an image with a spot of interest from being missed.

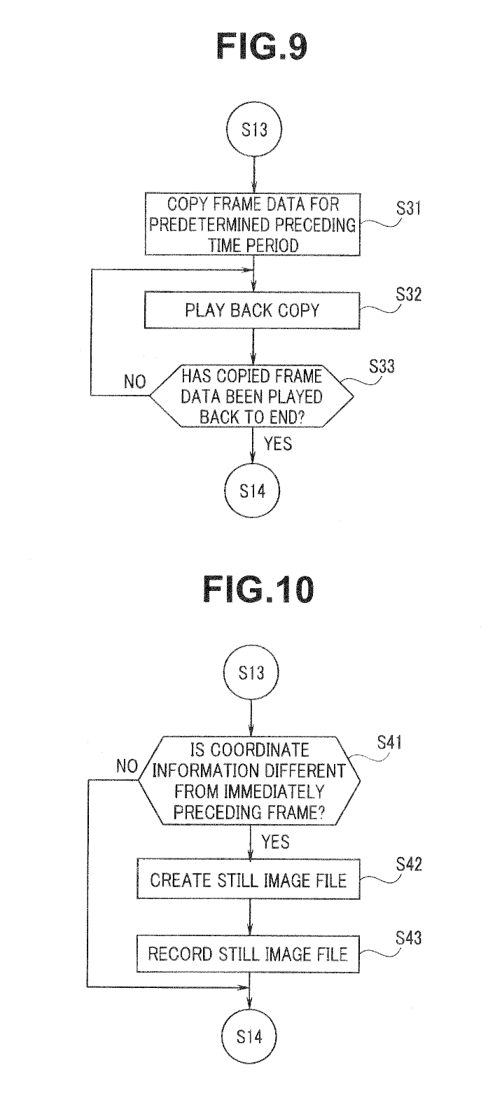

FIG. 9 is a flow chart showing a flow of a moving image playback process according to the second modification. The process in FIG. 9 is a process which is added and executed between S13 and S14 of the process in FIG. 4.

If it is judged in S12 that the acquired frame has a piece of coordinate information (YES in S12), the CPU 21 displays the indicator M2 (S13) and copies pieces of frame data for a predetermined time period from, e.g., 5 seconds before to a present time onto a predetermined area of the RAM 23 (S31).

The CPU 21 plays back each copied piece of frame data (S32) and plays back the copied pieces of frame data to an end.

The CPU 21 judges (S33) whether the copied pieces of frame data are played back to the end. If the copied pieces of frame data are not played back to the end (NO in S33), the CPU 21 continues to play back the copy (S32).

On the other hand, if the copied pieces of frame data are played back to the end (YES in S33), the process shifts to S14 to play back the frame with a piece of coordinate information and subsequent frames.

The process in FIG. 9 can be implemented by, for example, the CPU 21 constantly storing most recently played back pieces of frame data for a predetermined time period in the RAM 23 or another buffer memory and copying the stored pieces of frame data for the predetermined time period (e.g., most recent 5 seconds) onto the predetermined area of the RAM 23.

As described above, in the processes in S13 to S14, if there is a shift from playback of a frame not associated with a piece of coordinate information to playback of a frame associated with a piece of coordinate information during playback of a recorded moving image, a playback position is returned to a frame earlier by the predetermined time period than the frame not associated with a piece of coordinate information once, and playback is performed from the frame earlier by the predetermined time period.

Accordingly, a user can carefully view and confirm a photographed spot of interest while the indicator M2 is displayed in a superimposed manner from a position earlier by the predetermined time period than a frame including the spot of interest. The configuration is particularly effective in preventing coordinates designated at the time of moving image recording from being missed if a time period during which the coordinates are designated is short.

### Third Modification

In a third modification, a still image is recorded for a piece of frame data associated with a piece of coordinate information at the time of playback, and the work of capturing a still image for a spot of interest from a moving image is automated.

FIG. 10 is a flow chart showing a flow of a moving image playback process according to the third option. The process in FIG. 10 is a process which is added and executed between S13 and S14 of the process in FIG. 4.

If it is judged in S12 that the acquired frame is associated with a piece of coordinate information (YES in S12), the CPU 21 displays the indicator M2 (S13) and judges (S41) whether the piece of coordinate information of the current frame is different from a piece of coordinate information of an immediately preceding frame.

Note that although it is judged in S41 whether the piece of coordinate information of the acquired frame is different from the piece of coordinate information of the immediately preceding frame, if the pieces of coordinate information are little different (e.g., a difference between the pieces of coordinate information is not more than a predetermined value), i.e., two sets of coordinates are substantially same, the two pieces of coordinate information may be regarded as not different, as in the judgment in S21.

If the piece of coordinate information of the current frame is different from the piece of coordinate information of the immediately preceding frame (the case includes a case where the immediately preceding frame is not associated with a piece of coordinate information, and the current frame is associated with a piece of coordinate information) (YES in S41), the CPU 21 creates a still image file for the current frame (S42). The CPU 21 records the created still image file on the memory card 11 (S43).

Note that a still image file to be created may or may not include the piece of coordinate information associated with the current frame.

If the piece of coordinate information associated with the current frame is not different from the piece of coordinate information associated with the immediately preceding frame (NO in S41), no processing is performed.

As described above, the processes in S13 to S14 constitute a still image recording portion which, if there is a shift from playback of a frame not associated with a piece of coordinate information to playback of a frame associated with a piece of coordinate information during playback of a recorded moving image, generates a still image for the frame associated with a piece of coordinate information in the shift and records the still image on the memory card 11 serving as the recording portion.

Thus, according to the third modification, if a frame is judged to be a frame with a piece of coordinate information, a still image for the frame is automatically saved. The configuration is convenient.

### Fourth Modification

In a fourth modification, a moving image is recorded for a piece of frame data associated with a piece of coordinate information at the time of playback, and the work of capturing a moving image for a spot of interest is automated.

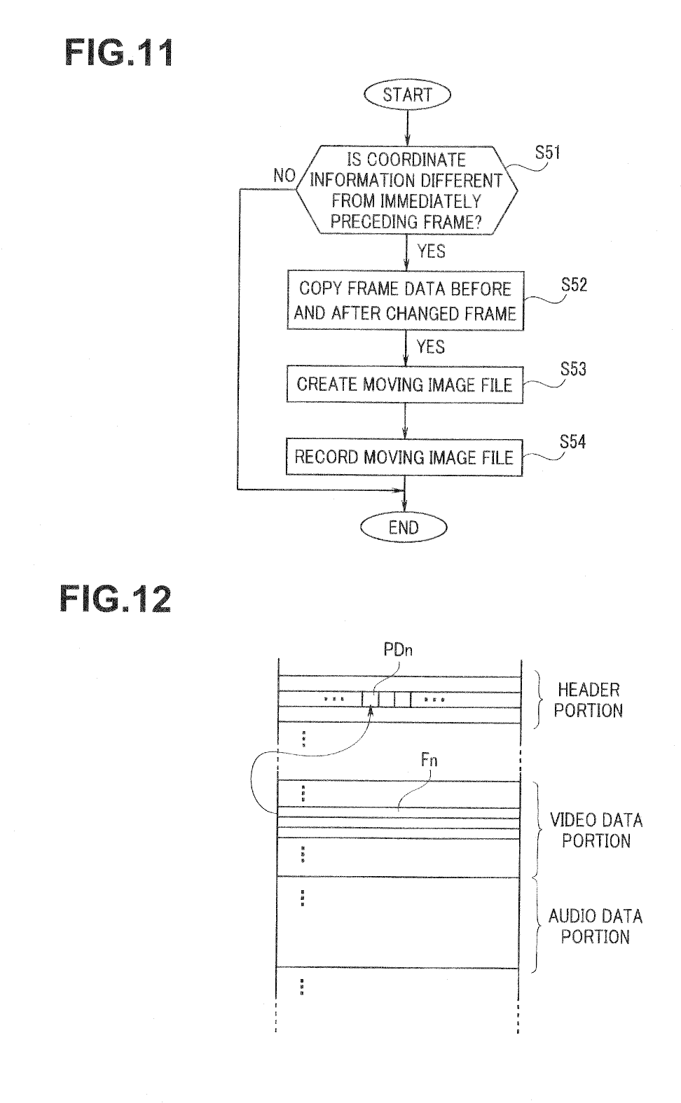

FIG. 11 is a flow chart showing an example of a flow of a moving image playback process according to the fourth modification. Note that the process in FIG. 11 is executed as so-called background processing when the CPU 21 plays back a moving image.

The CPU 21 sequentially reads out pieces of frame data of a moving file being played back from a first piece of frame data. The CPU 21 judges whether a current frame is different in coordinate information from an immediately preceding frame (S51).

Note that although it is judged in S51 whether a piece of coordinate information of the acquired frame is different from a piece of coordinate information of the immediately preceding frame, if the pieces of coordinate information are substantially not different (e.g., a difference between the pieces of coordinate information is not more than a predetermined value), i.e., two sets of coordinates are substantially same, the two pieces of coordinate information may be regarded as not different, as in the judgment in S21.

If the piece of coordinate information associated with the read-out frame is different from the piece of coordinate information associated with the immediately preceding frame (YES in S51), the CPU 21 copies pieces of frame data before and after the read-out frame onto the RAM 23 (S52). For example, pieces of frame data of duration of 5 seconds before the read-out frame and pieces of frame data of duration of 5 seconds after the read-out frame (duration of 10 seconds in total) are copied.

The CPU 21 creates a moving image file from the copied pieces of frame data (S53) and records the created moving file onto the memory card 11 (S54).

Note that a moving file to be created may or may not include a piece of coordinate information associated with each frame.

If the piece of coordinate information associated with the current frame is not different from the piece of coordinate information associated with the immediately preceding frame (NO in S51), no processing is performed.

As described above, the processes in S13 to S14 constitute a moving image recording portion which, if there is a shift from playback of a frame not associated with a piece of coordinate information to playback of a frame associated with a piece of coordinate information during playback of a moving image, generates a moving image including frames before and after the shift of duration of a predetermined time period and records the moving image on the memory card 11 serving as the recording portion.

Thus, according to the fourth modification, if a frame is judged to be a frame with a piece of coordinate information, a moving image including frames before and after the frame is automatically saved. The configuration is convenient.

### Fifth Modification

In a fifth modification, a trajectory of an indicator is displayed during playback of a moving image.

In each of the embodiment and modifications described above, the indicator M1 (or M2) indicates a spot of interest in each frame. Accordingly, the indicator M1 (or M2) moves in response to a change in a spot of interest.

However, in some cases, inspection is easier when a change in a position of a spot of interest is displayed as a trajectory.

For the reason, in the fifth modification, trajectories of the indicators M1 and M2 are displayed. Display of trajectories of the indicators M1 and M2 can be implemented by, for example, omitting the process of S7 in FIG. 2 and the process of S19 in FIG. 4, i.e., a process of erasing the indicator M1 (or M2) when the indicator is displayed. That is, trajectories of the indicators M1 and M2 are displayed by omitting the processes of S7 and S19 in FIGS. 2 and 4.

Note that display of a trajectory may be performed only at the time of moving image recording or playback.

As described above, a trajectory of a position detected in S2 is displayed on the basis of the position in at least one of the moving image recording process and the playback process. If the indicators M1 and M2 are dots, a trajectory of each dot is displayed as a figure drawn freehand.

Since the indicator M2 is displayed in a superimposed manner, indication of spots of interest is consequently reproduced and displayed as a trajectory of the indicator M2. The configuration allows a user to easily know a spot of interest.

### Sixth Modification

A sixth modification is configured such that a display position of an indicator is changeable during playback.

In some cases, a position of the indicator M2 attached to a played-back moving image may deviate from an appropriate position. In such a case, a user pauses a moving image, places the endoscope apparatus 1 into coordinate change mode for changing coordinates of the indicator M2. The user can select the indicator M2 with a cursor and move the indicator M2.

In coordinate change mode, when the user moves the selected indicator M2 and gives instructions for confirmation when the indicator M2 is at a desired position on a screen, a piece of coordinate information associated with a current frame is updated with a piece of coordinate information of the position as the movement destination.

FIG. 12 is a diagram for explaining association between a piece of frame data and a piece of coordinate information. A moving image data file includes a header portion, a video data portion, and an audio data portion, as shown in FIG. 12. As shown in FIG. 12, a piece of coordinate information associated with a frame Fn is stored in a storage area PDn of the header portion.

Accordingly, a user moves the indicator M2 corresponding to the frame Fn and gives instructions for confirmation when the indicator M2 is at a desired position on a screen in coordinate change mode, the CPU 21 senses the movement and confirmation and updates a piece of data in the storage area PDn of the header portion with a piece of coordinate information of the position after the change.

As described above, it is possible to pause playback at the time of playing back a recorded moving image and change a piece of coordinate information for a frame displayed during the pause.

Thus, according to the sixth modification, a display position of the displayed indicator M2 is revised to a more appropriate position and is recorded, which further improves inspection efficiency.

### Seventh Modification

A seventh modification is configured such that a still image can be temporarily displayed instead of a moving image during recording of the moving image, and a plurality of spots of interest can be designated for the still image.

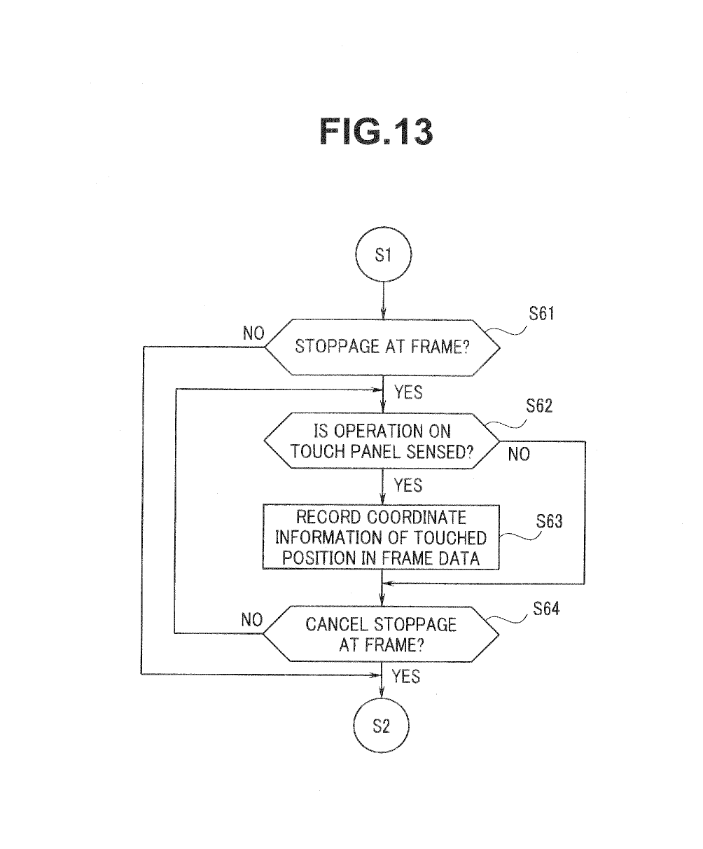

FIG. 13 is a flow chart showing an example of a flow of a moving image recording process according to the seventh modification. The process in FIG. 13 is a process to be added and executed between S1 and S2 of the process in FIG. 2.

After acquiring the piece of image data for the frame in S1, the CPU 21 judges (S61) whether instructions for stoppage at the frame are given by a user. Instructions for stoppage at the frame are given when the user selects a predetermined button displayed on the LCD 4.

If instructions for stoppage at the frame are given (YES in S61), the CPU 21 senses presence or absence of an operation on the touch panel 32 (S62). If an operation on the touch panel 32 is sensed (YES in S62), the CPU 21 records a piece of coordinate information of a touched position in a corresponding piece of frame data (S63). If instructions for stoppage at the frame are not given (NO in S61), no processing is performed.

If an operation on the touch panel 32 is not sensed (NO in S62) and after the piece of coordinate information of the touched position is recorded (S63), the CPU 21 judges whether stoppage at the frame is canceled (S64).

If stoppage at the frame is not canceled (NO in S64), the process shifts to S62. On the other hand, if stoppage at the frame is canceled (YES in S64), the process ends.

As described above, a plurality of pieces of coordinate information can be stored in association with each frame of a moving endoscope image at the time of recording of the moving image. If the indicators M1 and M2 are dots, a trajectory of each dot can also be displayed as a figure drawn freehand in each frame. Accordingly, a plurality of spots of interest can be indicated in one frame, which leads to improvement in inspection efficiency.

Note that although the seven modifications have been described above, the seven modifications can be simultaneously executed in any combination within an applicable range.

As described above, according to the endoscope systems of the embodiment and options described above, an endoscope system capable of efficiently confirming an inspection result can be implemented by displaying, at the time of playing back a moving endoscope image, a predetermined mark on the basis of a piece of coordinate information of a position on the moving image which is inputted at the time of moving image recording. This leads to a further reduction in inspection mistakes and a further reduction in labor required for inspection.

The present invention is not limited to the above-described embodiment, and various changes, alterations and the like may be made without departing from spirit and scope of the present invention.

What is claimed is:

1. An endoscope system comprising:
a position input detection portion which detects input of a position on a moving endoscope image to be recorded;
a coordinate information storage portion which stores a piece of coordinate information of the position detected by the position input detection portion in association with each of frames of the moving endoscope image;
a recording portion which records pieces of frame data of the moving image; and
a moving image playback portion which reads out the pieces of frame data of the moving image from the recording portion, judges whether each of the frames is associated with the piece of coordinate information, superimposes a predetermined first mark at the position on the basis of the piece of coordinate information if the frame is associated with the piece of coordinate information, and plays back the moving endoscope image.

2. The endoscope system according to claim 1, further comprising
an endoscope image display portion which displays a predetermined second mark on the basis of the piece of coordinate information of the position detected by the position input detection portion so as to be superimposed on the recorded moving endoscope image.

3. The endoscope system according to claim 1, wherein
the moving image playback portion plays back the moving endoscope image without superimposing the predetermined mark if the frame is not associated with the piece of coordinate information.

4. The endoscope system according to claim 1, wherein
the coordinate information storage portion stores the piece of coordinate information in the recording portion that records the moving endoscope image.

5. The endoscope system according to claim 1, wherein
the coordinate information storage portion stores the piece of coordinate information in association with each frame by writing the piece of coordinate information in the piece of frame data for the frame.

6. The endoscope system according to claim 1, wherein
the position input detection portion detects, as the position, a position designated by a touch panel or a pointing device on a screen of a display portion on which the endoscope image is displayed.

7. The endoscope system according to claim 1, wherein
if a skip playback command is inputted during playback of the recorded moving image, the moving image playback portion searches for a frame associated with the piece of coordinate information among frames which are later than a frame which is at a current playback position and changes a playback position from the current playback position to a position of the frame associated with the piece of coordinate information that is detected by the search.

8. The endoscope system according to claim 1, wherein
the moving image playback portion pauses playback if there is a shift from playback of a frame which is not associated with the piece of coordinate information to playback of a frame associated with the piece of coordinate information during playback of the recorded moving image and resumes playback from the paused frame associated with the piece of coordinate information if instructions to resume playback are given.

9. The endoscope system according to claim 1, wherein
the moving image playback portion returns to a frame which is earlier by a predetermined first time period than a frame not associated with the piece of coordinate information once and performs playback from the frame earlier by the predetermined first time period if there is a shift from playback of the frame not associated with the piece of coordinate information to playback of a frame which is continuous with the frame and is associated with the piece of coordinate information during playback of the recorded moving image.

10. The endoscope system according to claim 1, further comprising
a still image recording portion which, if there is a shift from playback of a frame which is not associated with the piece of coordinate information to playback of a frame associated with the piece of coordinate information during playback of the recorded moving image, generates a still image for the frame associated with the piece of coordinate information in the shift and records the still image on the recording portion.

11. The endoscope system according to claim 1, further comprising
a moving image recording portion which, if there is a shift from playback of a frame which is not associated with the piece of coordinate information to playback of a frame associated with the piece of coordinate information during playback of the recorded moving image, generates a moving image including frames which are of a duration of a predetermined second time period before and after the shift and records the moving image on the recording portion.

12. The endoscope system according to claim 1, wherein
at least one of the endoscope image display portion and the moving image playback portion displays a trajectory of the position on the basis of the position detected by the position input detection portion.

13. The endoscope system according to claim 1, wherein
the moving image playback portion can pause playback during playback of the recorded moving image and change the piece of coordinate information for a frame which is displayed during the pause.

14. The endoscope system according to claim 1, wherein
the coordinate information storage portion can store a plurality of the pieces of coordinate information in association with each frame of the moving endoscope image.

15. An image playback method in an endoscope system, comprising:
detecting input of a position on a moving endoscope image to be recorded;
storing a piece of coordinate information of the detected position in association with each of frames of the moving endoscope image; and
reading out pieces of frame data of the moving image from a recording portion which records the pieces of frame data of the moving image, judging whether each of the frames is associated with the piece of coordinate information, superimposing a predetermined first mark at the position on the basis of the piece of coordinate information if the frame is associated with the piece of coordinate information, and playing back the moving endoscope image.

16. An endoscope system comprising:
detection means for detecting input of a position on a moving endoscope image to be recorded;
coordinate information storage means for storing a piece of coordinate information of the position detected by the detection means in association with each of frames of the moving endoscope image;
recording means for recording pieces of frame data of the moving image; and
moving image playback means for reading out the pieces of frame data of the moving image from the recording means, judging whether each of the frames is associated with the piece of coordinate information, superimposing a predetermined first mark at the position on the basis of the piece of coordinate information if the frame is associated with the piece of coordinate information, and playing back the moving endoscope image.
